(19) United States
(12) Patent Application Publication
(10) Pub. No.: US 2024/0398919 A1
(43) Pub. Date: Dec. 5, 2024

(54) EPITOPE PEPTIDE FOR TUMOR-ASSOCIATED ANTIGEN AND USE THEREOF

(71) Applicant: SHENZHEN INSTITUTES OF ADVANCEDTECHNOLOGY CHINESE ACADEMY OF SCIENCES, Shenzhen (CN)

(72) Inventors: Jiandong HUANG, Shenzhen (CN); Meiling JIN, Shenzhen (CN); Shuzhen LIU, Shenzhen (CN); Mingshuo ZHAN, Shenzhen (CN)

(73) Assignee: SHENZHEN INSTITUTES OF ADVANCEDTECHNOLOGY CHINESE ACADEMY OF SCIENCES, Shenzhen (CN)

(21) Appl. No.: 18/810,833

(22) Filed: Aug. 21, 2024

Related U.S. Application Data
(63) Continuation of application No. PCT/CN2023/141695, filed on Dec. 25, 2023.

(30) Foreign Application Priority Data
May 12, 2023 (CN) 202310534458.4

(51) Int. Cl. A61K 39/00; A61P 37/04; C07K 14/47

(52) U.S. Cl. CPC A61K 39/0011; A61P 37/04; C07K 14/4748

(57) ABSTRACT

The present disclosure relates to epitope peptides for tumor-associated antigens and use thereof. The epitope peptides for tumor-associated antigens are derived from embryonic stem cells, and NUF-2. Studies have found that among embryonic stem cell-derived tumor-associated antigens selected from at least one of CENPM, IQGA3-1, IQGA3-2, KIF4A-1, KIF4A-2, and NUF-2, the tumor-associated antigens KIF4A and NUF-2 expressed by embryonic stem cells (ESCs) can effectively inhibit the growth of bladder cancer, and the tumor-associated antigens CENPM, NUF-2, and IQGA3 expressed by the ESCs can strongly stimulate the immune response of specific T cells, which is manifested by stimulating peptide-specific CTL to secrete high levels of IFN-γ and inhibiting tumor growth, and the epitope peptides for tumor-associated antigens can be used in the preparation of a medicament for inhibiting the growth of tumor cells or stimulating immune cells to produce a T cell response.

### CROSS-REFERENCE TO RELATED APPLICATIONS

The application claims priority to Chinese patent application No. 202310534458.4, filed on May 12, 2023, the entire contents of which are incorporated herein by reference.

### SEQUENCE LISTING

The sequence listing xml file submitted herewith, named “EPITOPEPEPTIDEFORTUMORASSOCIATEDANTIGENANDUSETHEREOF.xml”, created on May 9, 2023, and having a file size of 18,214 bytes, is incorporated by reference herein.

### TECHNICAL FIELD

The present disclosure belongs to the field of biotechnology and relates to epitope peptides for tumor-associated antigens and use thereof.

### BACKGROUND

According to the latest statistics from the International Agency for Research on Cancer (IARC) under the World Health Organization, bladder cancer is the tenth most common type of cancer in the world. Each year, about 600,000 people worldwide are diagnosed with bladder cancer, and more than 200,000 people die from this disease. Bladder cancer is one of the most challenging cancers to diagnose and treat. Its diagnosis relies mainly on cystoscopy, which is an invasive and costly way. Currently, the traditional treatment methods for cancer include surgery, radiotherapy, and chemotherapy. Although the conventional treatment methods can effectively cure cancer, damage to the immune system is large and the needs of individualized diagnosis and treatment cannot be met. Emerging immunotherapies, represented by vaccines, monoclonal antibodies, and adoptive T-cell therapies, has made great progress in the past few decades, better making up for the disadvantages of traditional therapies. A large number of studies have entered the stage of clinical trial evaluation. A single or combined solution may provide a more effective solution for patients.

Unlike traditional prophylactic vaccines, which stimulate the body to produce an immune response from exogenous antigens, cancer vaccines can target endogenous intracellular antigens and may trigger new tumor-specific immune responses, thereby exerting therapeutic effects. The emergence of therapeutic cancer vaccines dates back to the early 20th century, which used killed streptococci and serratia to treat malignant tumors. Today, cancer vaccines have become a promising candidate therapeutic strategy for solid tumor immunotherapy that stimulates anti-tumor immunity through tumor antigens delivered in the form of whole cells, polypeptides, nucleic acids, etc. The tumor antigens are divided into Tumor-Specific Antigens (TSAs) and Tumor-Associated Antigens (TAAs). The difference is that the former is only an antigen that is expressed in tumor cells and not in normal cells, while the latter is present in both tumor cells and normal cells, but is an antigen that is highly expressed in the tumor cells. The cancer vaccines are divided into two categories, predefined antigens and anonymous antigens, according to whether antigens are defined before treatment. The predefined antigens are further subdivided into two categories, i.e., personalized antigens and shared antigens according to their expression frequency in patients with the same tumor type. Shared vaccines are widely used in patient populations and can be evaluated by standard detection methods (cytology, immunohistochemistry, flow cytometry, etc.), targeting both TSAs and TAAs. The cancer vaccines work primarily through recognition and uptake of tumor antigens by Antigen-Presenting Cells (APCs), and presentation at HLA-1 to CD8+ T cells (also called cytotoxic T lymphocytes), which subsequently secrete various cytokines (e.g., IFN-γ) to induce an immune response in the body and kill the tumor. In summary, due to the weak immunogenicity of the current tumor vaccines, the immune escape of tumor cells leads to poor tumor vaccine efficacy, resulting in poor tumor inhibition effects.

### SUMMARY

Based on this, it is necessary to provide epitope peptides for tumor-associated antigens that can be used in the preparation of a medicament for inhibiting the growth of tumor cells or stimulating immune cells to produce a specific T-cell response and use thereof.

Provided are epitope peptides for tumor-associated antigens, derived from embryonic stem cells, wherein the tumor-associated antigens are selected from at least one of CENPM, IQGA3-1, IQGA3-2, KIF4A-1, KIF4A-2, and NUF-2.

Studies have found that among embryonic stem cell-derived tumor-associated antigens selected from at least one of CENPM, IQGA3-1, IQGA3-2, KIF4A-1, KIF4A-2, and NUF-2, the tumor-associated antigens KIF4A and NUF-2 expressed by embryonic stem cells (ESCs) can effectively inhibit the growth of bladder cancer, and the tumor-associated antigens CENPM, NUF-2, and IQGA3 expressed by the ESCs can strongly stimulate the immune response of specific T cells, which is manifested by stimulating peptide-specific CTL to secrete high levels of IFN-γ and inhibiting tumor growth, playing a certain therapeutic role, and the epitope peptides for tumor-associated antigens can be used in the preparation of a medicament for inhibiting the growth of tumor cells or stimulating tumor cells to produce a T-cell response. The inhibitory effect of the vaccine on bladder cancer in mice has been verified by in vivo experiments.

In some examples, the epitope peptides for tumor-associated antigens include one of peptide fragments with amino acid sequences as shown in SEQ ID No. 1-SEQ ID No. 10.

In some examples, the epitope peptides for tumor-associated antigens include one of peptide fragments with amino acid sequences as shown in SEQ ID No. 11-SEQ ID No. 20.

Provided is use of the epitope peptides for tumor-associated antigens described above in the preparation of a medicament for inhibiting the growth of tumor cells and/or stimulating tumor cells to produce a T-cell response.

In some examples, the tumor includes at least one of bladder cancer, esophageal cancer, kidney cancer, liver cancer, lung cancer, ovarian cancer, pancreatic cancer, prostate cancer, gastric cancer, and uterine cancer.

Provided is a medicament for treatment of tumors, including: at least one of the epitope peptides for tumor-associated antigens described above.

In some examples, the epitope peptide for tumor-associated antigens in the medicament for treatment of tumors includes a peptide fragment having an amino acid sequence as shown in SEQ ID No. 10 or a peptide fragment having an amino acid sequence as shown in SEQ ID No. 20.

In some examples, the epitope peptide for tumor-associated antigens in the medicament for treatment of tumors includes a first peptide fragment including at least one of an amino acid sequence as shown in SEQ ID No. 9 and an amino acid sequence as shown in SEQ ID No. 19, and a second peptide fragment including at least one of an amino acid sequence as shown in SEQ ID No. 8 and an amino acid sequence as shown in SEQ ID No. 18.

In some examples, the medicament for treatment of tumors further includes an adjuvant, including at least one of CpG oligodeoxynucleotide and Poly IC.

In some examples, the medicament for treatment of tumors further includes a pharmaceutically acceptable carrier and/or excipient.

Provided is use of antigenic epitope peptides derived from embryonic stem cells in the preparation of a medicament for inhibiting the growth of tumor cells and/or stimulating tumor cells to produce a T-cell response, wherein tumor-associated antigens are selected from at least one of CENPM, IQGA3-1, IQGA3-2, KIF4A-1, KIF4A-2, and NUF-2.

### BRIEF DESCRIPTION OF DRAWINGS

FIG. 1 is a flow chart of test operations in Example 1;

FIG. 2 is a graph showing the detection results of a tumor growth inhibition assay of antigenic epitope peptides derived from various genes tested;

FIG. 3 is a graph showing the detection results of the inhibitory effect of mixed effective antigenic epitope peptide vaccines in Example 1 on tumor growth;

FIG. 4 is a graph showing the detection results of the effect of mixed peptides on immune cells in blood;

FIGS. 5A and 5B is a graph showing the detection results of the ability of specific T cells to secrete IFN-γ induced by tumor antigen epitope peptides.

### DETAILED DESCRIPTION OF THE EMBODIMENTS

In order that the above objects, features and advantages of the present disclosure can be more clearly understood, the specific embodiments of the present disclosure will be described in detail with reference to the examples and the accompanying drawings. Numerous specific details are set forth in the following description in order to provide a thorough understanding of the present disclosure. However, the present disclosure can be implemented in many ways other than those described herein, those skilled in the art may make similar modifications without violating the connotation of the present disclosure, and therefore the present disclosure is not limited by the specific embodiments disclosed below.

The present disclosure provides epitope peptides for tumor-associated antigens, derived from embryonic stem cells, wherein the tumor-associated antigens are selected from at least one of CENPM, IQGA3-1, IQGA3-2, KIF4A-1, KIF4A-2, and NUF-2.

The potential of stem cell vaccines to treat cancer comes from the early observation that embryonic/fetal tissue immunity results in rejection of transplanted tumors in animals, and some subsequent studies further demonstrate that inoculation of embryonic materials in animals can induce cellular and humoral immunity against transplantable tumors and carcinogen-induced tumors. The similarity of tumor cell and embryonic cell antigens (i.e., shared antigens) is a hypothetical basis for their use as a vaccine to treat cancer. After 3 weeks of continuous inoculation with Hepatic Stem Cells (HSCs) or Embryonic Stem Cells (ESCs), liver cancer cells Hepa 1-6 were subcutaneously inoculated at 1 week (Group 1) and 4 weeks (Group 2) after inoculation, respectively, in the experimental groups, no tumor formation was seen in the HSC vaccine-immunized mice within one week, only 10% of the ESC vaccine-immunized mice developed tumors, whereas the mice in the control group had a tumor formation rate of 60%, and the long-term memory response was measured in approximately the same situation (Group 2) as that in (Group 1). The same period of experiments showed that in a subcutaneous liver cancer mouse model, 80% of mice vaccinated with HSCs completely cleared the tumor burden, but 40% of mice vaccinated with ESCs did not develop tumors that increased over time, the data supporting the excellent performance of stem cells as a prophylactic or therapeutic cancer vaccine. However, the development of embryonic/fetal materials into vaccines for clinical use may have certain limitations, such as ethical aspects, tumorigenicity and alloimmunity. Autologous Induced Pluripotent Stem Cells (iPSCs) can also be used directly as a vaccine after irradiation. Its emergence is overcoming the above limitations. It is worth noting that single iPSC immunization cannot play a strong anti-tumor effect, while CpG as an adjuvant in combination solves this problem, and its anti-cancer effect has been verified in breast cancer, melanoma and mesothelioma.

Centromere protein M (CENPM) is encoded by a gene CENPM and is involved in kinetochore protein assembly and chromosome segregation, and plays an important role in a cell cycle. Recent research advances have focused on the association of this protein with carcinogenesis. CENPM promotes tumorigenesis through multiple pathways such as p53 and mTOR/p70S6k. Kinetochore protein (NUF2) is encoded by NUF2, is associated with centromeres, and is involved in chromosome segregation. Similarly, upregulation of NUF2 also promotes tumorigenesis, and changes in NUF2 levels have effects on cell proliferation, migration, and invasion. A member of the kinesin superfamily, a chromosome-associated molecular motor (KIF4A), is encoded by KIF4A, is involved in intracellular transport, and is responsible for maintenance of cell physiological morphology, and in addition, it is also involved in chromosome aggregation and segregation during mitosis. Similarly, changes in KIF4A levels have also been associated with tumor development. To sum up, they have the potential to serve as prognostic markers for cancer, however, there are relatively few reports on their use as antigen polypeptides.

Studies have found that among embryonic stem cell-derived tumor-associated antigens selected from at least one of CENPM, IQGA3-1, IQGA3-2, KIF4A-1, KIF4A-2, and NUF-2, the tumor-associated antigens KIF4A and NUF-2 expressed by embryonic stem cells (ESCs) can effectively inhibit the growth of bladder cancer, the tumor-associated antigens CENPM, NUF-2, and IQGA3 expressed by the ESCs can strongly stimulate the immune response of specific T cells, which is manifested by stimulating peptide-specific CTL to secrete high levels of IFN-γ and inhibiting tumor growth, playing a certain therapeutic role, and the tumor-associated antigens can be used in the preparation of a medicament for inhibiting the growth of tumor cells or stimulating tumor cells to produce a T-cell response. The inhibitory effect of the vaccine on bladder cancer in mice has been verified by in vivo experiments.

In some examples, the epitope peptides for tumor-associated antigens include one of peptide fragments with amino acid sequences as shown in SEQ ID No. 1-SEQ ID No. 10.

In some examples, the epitope peptides for tumor-associated antigens include one of peptide fragments with amino acid sequences as shown in SEQ ID No. 11-SEQ ID No. 20. The peptide fragments with the amino acid sequences as shown in SEQ ID No. 11-SEQ ID No. 20 are extension peptides of the epitopes with the amino acid sequences as shown in SEQ ID No. 1-SEQ ID No. 10, and the two are basically the same in function, since a cleavage process is required during presentation of antigen epitopes by dendritic cells, the extension peptides including the peptide segments with the amino acid sequences as shown in SEQ ID No. 1-SEQ ID No. 10 are synthesized during actual synthesis.

An embodiment of the present disclosure also provides a medicament for treatment of tumors, including at least one of the epitope peptides for tumor-associated antigens described above.

Traditional methods, such as surgery and chemoradiotherapy, are invasive and have good effects on unspread tumors in an early stage, but cause undifferentiated and irreversible damage to the human body and the human immune system, increasing the risk of infection with other diseases. Immune checkpoint inhibitors improve the prognosis of chemotherapy intolerant populations, but face the problem of low overall patient response rates in clinical applications and the risk of inducing fatal immune-related side effects. Adoptive T cell therapy (e.g., Chimeric Antigen Receptor T-Cell, CAR-T) can quickly induce the body's immune response while bringing strong toxicity, and in addition, the time cost and high cost of transforming reinfusion, and off-target and antigen escape also limit the application of this technology. Cancer vaccines have the advantages of high safety and low side effects due to their clear mechanism of action and known antigen targets. The present disclosure aims to expand a preparation process of a stem cell-based cancer vaccine epitope identification and system, accelerating the construction of a vaccine discovery platform.

Studies have found that among embryonic stem cell-derived tumor associated antigens selected from at least one of CENPM, IQGA3-1, IQGA3-2, KIF4A-1, KIF4A-2, and NUF-2, the tumor-associated antigens KIF4A and NUF-2 expressed by embryonic stem cells (ESCs) can effectively inhibit the growth of bladder cancer, the tumor-associated antigens CENPM, NUF-2, and IQGA3 expressed by the ESCs can strongly stimulate the immune response of specific T cells, which is manifested by stimulating peptide-specific CTL to secrete high levels of IFN-γ and inhibiting tumor growth, playing a certain therapeutic role, and the medicament for treatment of tumors prepared from the epitope peptide for tumor-associated antigens can inhibit the growth of tumor cells or stimulate tumor cells to produce a T-cell response. The inhibitory effect of the vaccine on bladder cancer in mice has been verified by in vivo experiments.

In some examples, the tumor includes at least one of bladder cancer, esophageal cancer, kidney cancer, liver cancer, lung cancer, ovarian cancer, pancreatic cancer, prostate cancer, gastric cancer, and uterine cancer.

In some examples, the epitope peptide for tumor-associated antigens in the medicament for treatment of tumors includes a peptide fragment having an amino acid sequence as shown in SEQ ID No. 10 or a peptide fragment having an amino acid sequence as shown in SEQ ID No. 20. After experimental verification, the epitope peptide for tumor-associated antigens in this medicament can cause a relatively strong T-cell response and a relatively significant tumor-inhibiting effect, and can be used as an anti-tumor drug.

In some examples, the epitope peptide for tumor-associated antigens in the medicament for treatment of tumors includes a first peptide fragment including at least one of an amino acid sequence as shown in SEQ ID No. 9 and an amino acid sequence as shown in SEQ ID No. 19, and a second peptide fragment including at least one of an amino acid sequence as shown in SEQ ID No. 8 and an amino acid sequence as shown in SEQ ID No. 18. After experimental verification, the epitope peptide for tumor-associated antigens in this medicament has a relatively significant tumor-inhibiting effect.

In some examples, the medicament for treatment of tumors further includes an adjuvant, including at least one of CpG oligodeoxynucleotide and Poly IC. With the aid of the above adjuvant, the anti-tumor effect of the epitope peptides for tumor-associated antigens or the effectiveness of treating tumors can be increased. Wherein the tumor may be bladder cancer.

In some examples, the medicament for treatment of tumors further includes a pharmaceutically acceptable carrier and/or excipient. The pharmaceutically acceptable carrier and/or excipient includes, for example, a solvent, which may be, for example, DPBS. It should be noted that without being limited to the above pharmaceutically acceptable carrier and/or excipient, corresponding pharmaceutically acceptable carriers and/or excipients may be selected as needed. In some examples, the medicament for treatment of tumors is a vaccine.

In the medicament for treatment of tumors, a vaccine prepared from the tumor-associated antigens KIF4A-1, KIF4A-2, and NUF-2 expressed by ESCs can effectively inhibit the growth of bladder cancer, and CENPM and NUF-2 can strongly stimulate the immune response of specific T cells, which is manifested by stimulating peptide-specific CTL to secrete high levels of IFN-γ and inhibiting tumor growth, playing a certain therapeutic role.

Further, in the medicament for treatment of tumors, individual peptide fragments CENPM, NUF-2, KIF4A-1, and KIF4A-2 having a cancer-inhibiting effect are prepared into a polypeptide to be used as a vaccine to immunize mice, and the combination shows a good cancer-inhibiting effect. Moreover, the combination of selected peptide fragments and the adjuvant CpG oligodeoxynucleotide (CPG-ODN) has certain effectiveness in the treatment of bladder cancer. Embryonic stem cell-derived peptide fragments can be used in the preparation of tumor therapeutic polypeptide vaccines.

The present disclosure provides several tumor-associated antigens (TAAs) expressed by embryonic stem cells (ESCs) as potential therapeutic TAAs and effective epitopes that enable the preparation of shared vaccines that effectively inhibit the growth of bladder cancer, have high safety, low toxic side effects, and high specificity.

Specific examples are provided below.

Reagents and equipment used in the examples are routinely selected in the art unless otherwise specified. Experimental methods of which specific conditions are not specified in the examples are generally implemented according to conventional conditions, such as those described in the literature or books or by methods recommended by the manufacturer of the kit. The reagents used in the examples are all commercially available.

In the following examples, unless otherwise specified, proteins screened in Example 1 and epitope peptide sequences thereof are detailed in Table 1.

TABLE 1
Proteins screened in Example 1 and epitope peptide sequences thereof peptide

| Serial | | Epitope peptide | Population | |
|---|---|---|---|---|
| number | Protein | sequence | coverage (%) | Extension peptide sequence |
| 1 | CENPM | VHLAKSLPL | 0.5133 | EDCASELKVHLAKSLPLPSSVNRPR |
| | | (shown in SEQ ID | | (shown in SEQ ID No. 11) |
| | | No. 1) | | |
| 2 | IQGA3-1 | RYFDALLKL | 0.7862 | EVEGENAQRYFDALLKLRQERGMGE |
| | | (shown in SEQ ID | | (shown in SEQ ID No. 12) |
| | | No. 2) | | |
| 3 | IQGA3-2 | VAPRYHLLL | 0.9076 | GLDDVSLPVAPRYHLLLVAAKRQKA |
| | | (shown in SEQ ID | | (shown in SEQ ID No. 13) |
| | | No. 3) | | |
| 4 | IQGA3-3 | AYYFHLQTF | 0.747 | QHDMKDGTAYYFHLQTFQGIWEQPP |
| | | (shown in SEQ ID | | (shown in SEQ ID No. 14) |
| | | No. 4) | | |
| 5 | IQGA3-4 | AYQHLFYLL | 0.5765 | KEKRQKLEAYQHLFYLLQTQPIYLA |
| | | (shown in SEQ ID | | (shown in SEQ ID No. 15) |
| | | No. 5) | | |
| 6 | IQGA3-5 | LHYTAAQLL | 0.6873 | GKGKKQPSLHYTAAQLLEKGVLVEI |
| | | (shown in SEQ ID | | (shown in SEQ ID No. 16) |
| | | No. 6) | | |
| 7 | KIFC1 | WTYSFVASYV | 0.5024 | AQELSGQGWTYSFVASYVEIYNETV |
| | | (shown in SEQ ID | | (shown in SEQ ID No. 17) |
| | | No. 7) | | |
| 8 | KIF4A-1 | VFNTAVAPL | 0.524 | DPSTEQEEVENTAVAPLIKGVFKGY |
| | | (shown in SEQ ID | | (shown in SEQ ID No. 18) |
| | | No. 8) | | |
| 9 | KIF4A-2 | TSFFSGCSPI | 0.666 | GKKKKRALASNTSFFSGCSPIEEEA |
| | | (shown in SEQ ID | | (shown in SEQ ID No. 19) |
| | | No. 9) | | |
| 10 | NUF-2 | FYMMPVNSEVM | 0.6813 | YGIRLEHFYMMPVNSEVMYPHLMEG |
| | | (shown in SEQ ID | | (shown in SEQ ID No. 20) |
| | | No. 10) | | |

In Table 1, an extension peptide includes an epitope peptide sequence, and is functionally the same as the epitope peptide, since a cleavage process is required during presentation of antigen epitopes by dendritic cells, the extension peptide including the epitope peptide is synthesized during actual synthesis; and population coverage refers to the proportion of people in a population who possess at least one HLA allele that can bind to a peptide fragment.

### Example 1

The main contents of this example were as follows: a predictive immunoinformatics algorithm was used to predict shared genes that were highly expressed in ESCs and tumor cells but lowly expressed in normal tissues, and predict antigenic epitope peptides of CD8+ T cells with high expression of these genes, and CENPM, KIF4A-1, KIF4A-2, and NUF-2 were selected as potential therapeutic TAAs and effective epitopes. Vaccines based on these antigens CENPM, KIF4A-1, KIF4A-2, and NUF-2 were designed, and the continuous inhibitory effect of these antigens on tumor growth in a bladder cancer mouse model was verified, and the immune response of antigen-specific T cells was evaluated by an enzyme-linked immunospot assay (ELISPOT).

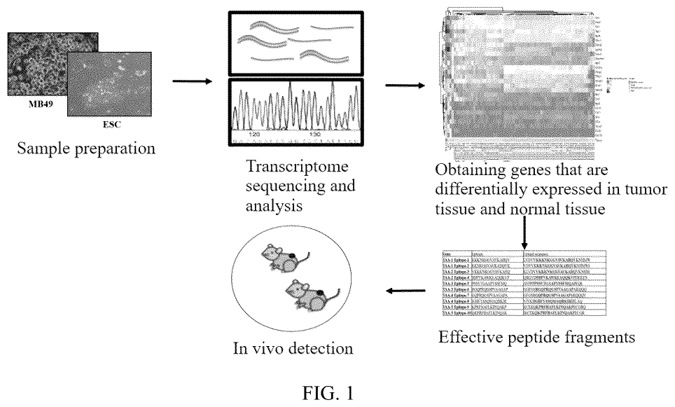

As shown in FIG. 1 (FIG. 1 is a flow chart of test operations in this example), a specific operation process of this example was as follows:

(1) Screening of therapeutic TAAs and effective epitopes:

By RNA-seq sequencing of an ESC (129) cell line, a bladder cancer cell line (MB49), a liver cancer cell line (ML-1) and a lung cancer cell line (LLC), the expressed genes were compared with the cancer cell line MB49 and healthy tissues, and shared genes highly expressed in ESCs but lowly expressed in normal tissues were screened as potential therapeutic TAAs.

By transcriptome sequencing and analysis of mouse tumor cells and embryonic stem cells, genes that are highly expressed in both tumor cells and embryonic stem cells were obtained, numbered 1-10. After literature research and in vitro experiment verification, effective genes were screened, including four genes, i.e., Pep-1 (cenpm), Pep-8 (kif-4a-1), Pep-9 (kif-4a-2), and Pep-10 (nuf-2). Antigenic epitopes of these genes were obtained through analysis. At the same time, extension peptides containing the antigenic epitope peptides of these genes were synthesized in cooperation with GL Biochem (Shanghai) Co., Ltd.

(2) Preparation and therapeutic ability evaluation of tumor vaccines:

Cancer cells and animal model: MB49 bladder cancer cells were grown in DMEM containing 10% FBS (Fetal Bovine Serum, Gibco, 10099141C) and 1% PS (Penicillin-Streptomycin-Glutamine, Gibco, 10378016). 3×10⁵ MB49 cancer cells were resuspended in 100 μL of DPBS and injected subcutaneously into a lower back of male C57BL/6 mice (6-8 weeks old), respectively to construct a mouse bladder cancer treatment model. After the initial implantation of tumors, the mice developed tumors around 5 days, and after tumor formation, the tumor growth was monitored every other 3 days, and a long diameter (a) and a short diameter (b) of the tumor were measured with a vernier caliper, and the size of each solid tumor was calculated according to the solid tumor size V=axbxb/2, and a tumor growth curve was plotted.

Vaccine preparation and periodic vaccination: tumor-bearing mice were randomly divided into: a DPBS group, a CpG group, a CpG+CENPM group, a CpG+IQGA3-1+IQGA3-2 group, a CpG+KIF4A-1+KIF4A-2 group, and a CpG+NUF-2 group, with 6 mice in each group. Each vaccine was prepared according to the groups with DPBS (phosphate buffered saline) as a solvent and CpG ODN 1826 as a vaccine adjuvant. The final concentration of CpG ODN 1826 was 10 μM, and the final concentration of each peptide fragment was 100 μg/100 μL. DPBS was used to make up to 100 μL, and the vaccines were injected subcutaneously into the hindneck in the back of the mice. The first vaccination was performed on Day 3, and the same vaccine was inoculated every other 3 days thereafter, with a total of 7 times.

Isolate splenocytes: Spleens were gently mashed and filtered through a 70-μm strainer (Falcon, Corning, Germany), and then collected into a 15 mL centrifuge tube. After adding 1 mL erythrocyte lysis buffer and washing in PBS, cells were collected for ELISPOT assay.

Isolation of Tumor Infiltrating Lymphocytes (TIL): solid tumors were dissected after the mice were sacrificed under anesthesia on Day 26. The tumors were cut into pieces and suspended in 5 mL of a digestive juice (50 mL HBSS containing 2 mg/mL Collagenase A and 50 units/mL DNase I) to be incubated at 37° C. at 180 rpm in a water bath for 20 min. Digested tumor pieces were collected into a 50 mL centrifuge tube through a 100 μm cell sieve, and digestion was stopped with a wash buffer containing EDTA. Cells were isolated by centrifugation with 40% and 80% percoll at 400 g for 25 min. Lymphocytes in a middle layer were collected, then washed with DPBS and counted for later use in ELISPOT.

Enzyme-linked immunospot assay (ELISPOT): splenocytes were isolated according to the above method and were co-cultured with different polypeptides (10 μg) at 5×10⁵/well for 20 h. The size and number of IFN-γ positive spots were calculated by using Adobe Photoshop CS6 software.

Data analysis: data were analyzed by using GraphPad Prism and all values in histograms and curves are expressed as mean+SEM. Differences between groups were evaluated by using Ordinary one-way ANOVA or Unpaired t test. * P<0.05, ** P<0.01, *** P<0.001, and **** P<0.0001.

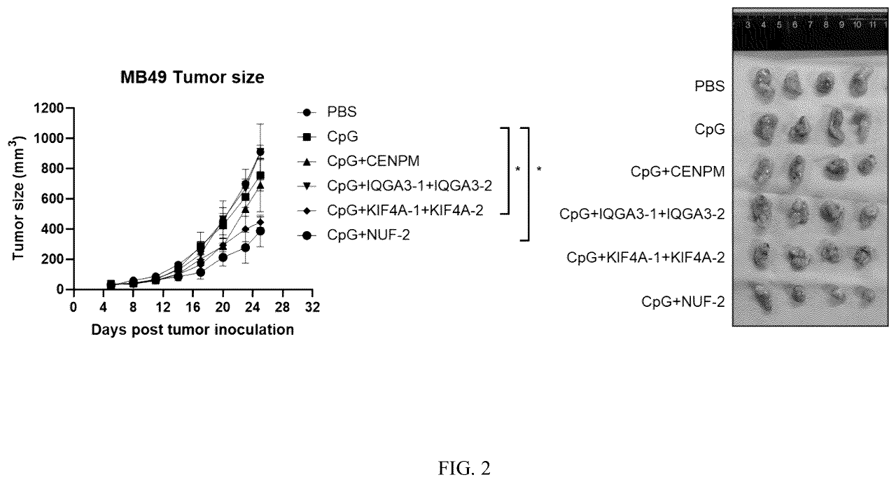

The experimental results are shown in FIGS. 2 to 5.

FIG. 2 is a graph showing the detection results of a tumor growth inhibition assay of antigenic epitope peptides derived from various genes tested. In FIG. 2, all mice were injected subcutaneously with 3×10⁵ MB49 bladder cancer cells on Day 0, immunized with peptide fragments on day 3, 6, 9, 12, 15, 18, and 21, and euthanized on day 26. Each mouse in the experimental groups was subcutaneously injected with synthesized epitope peptides and the adjuvant CpG, with a total injection amount of 100 μl per mouse, and the amount of CpG was 10 μM. The tumor size in the mice was detected every 3 days. As can be seen from FIG. 2, the tumor size in the mice in the KIF4A-1/−2 group and the NUF-2 group was significantly inhibited after immunization with different peptides, CENPM had a partial tumor growth inhibitory effect, and the inhibitory effect of the vaccines in the other groups on bladder cancer was not obvious.

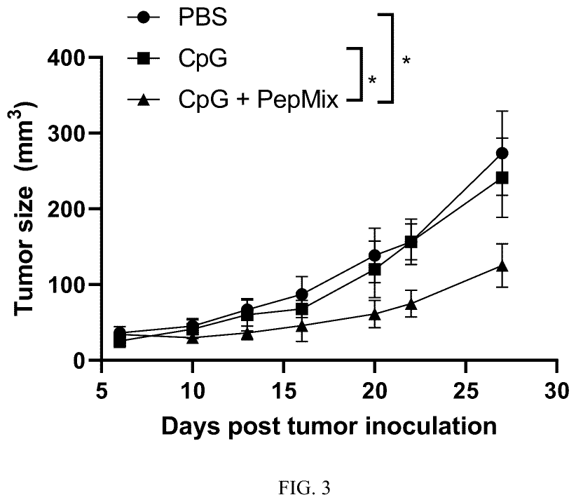

FIG. 3 is a graph showing the detection results of testing the inhibitory effect of mixed peptide vaccines on tumor growth after vaccines are prepared by mixing the effective antigenic epitope peptides. In FIG. 3, all mice were injected subcutaneously with 3×10⁵ MB49 bladder cancer cells on Day 0, immunized with peptide fragments on day 3, 6, 9, 12, 15, 18, and 21, and euthanized on day 26. Each mouse in the experimental groups was subcutaneously injected with synthesized epitope peptides and the adjuvant CpG, peptide fragments of vaccines including KIF4A-1/−2, NUF-2 and CENPM dissolved in 100 μL of DPBS, and the amount of the adjuvant CpG was 10 μM, with a total injection amount of 100 μl per mouse. The tumor size in the mice was detected every 3 days. As can be seen from FIG. 3, the mixed peptide fragment group showed significant tumor growth inhibition after immunization with different peptide fragments.

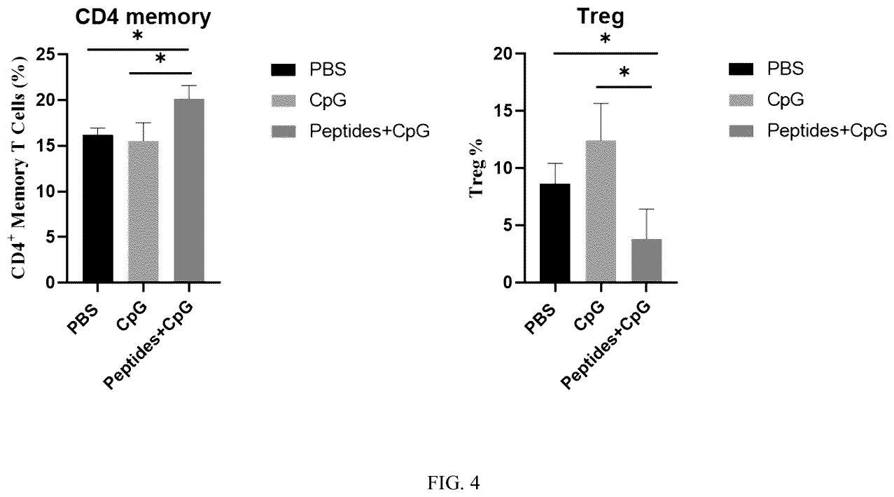

FIG. 4 is a graph showing the detection results of the effect of mixed peptide fragments on immune cells in blood. In FIG. 4, all mice were injected subcutaneously with 3×10⁵ MB49 bladder cancer cells on Day 0, immunized with mixed peptide fragments+adjuvant on day 3, 6, 9, 12, 15, 18, and 21, and were euthanized on day 26. Peripheral blood was taken when the mice were sacrificed, antibodies for CD45, CD4, CD44, CD25, and FOXP3 were added into 100 μL of the blood for flow cytometry detection, and the changes in the proportion of CD4 memory cells and Treg cells in the blood were analyzed. As can be seen from FIG. 4, the proportion of memory T cells was significantly upregulated and the Treg cells were significantly inhibited after immunization with mixed peptide fragments.

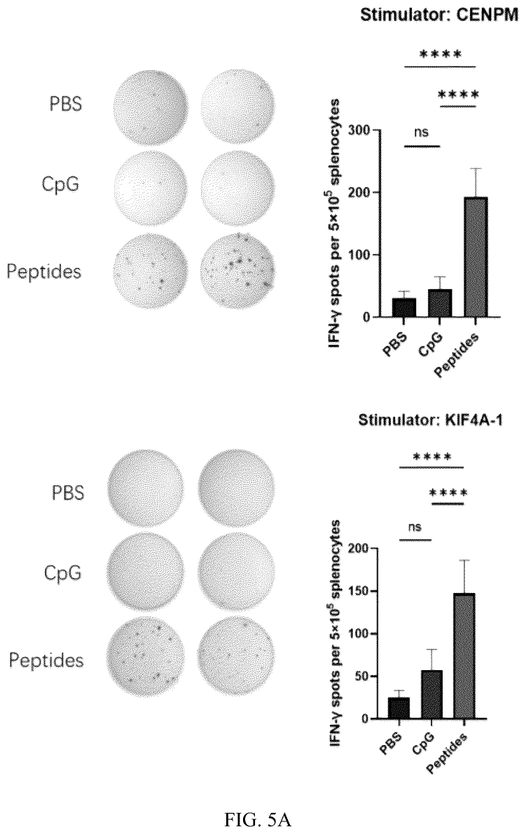

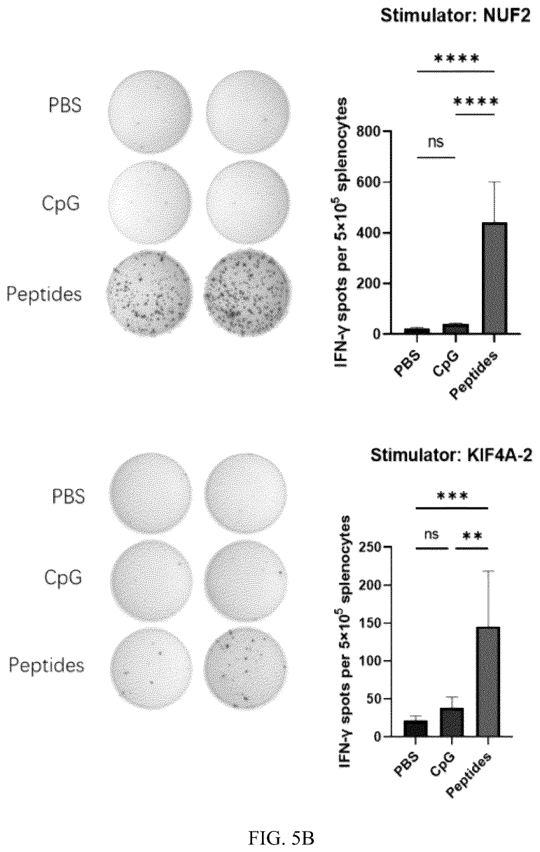

FIGS. 5A and 5B is a graph showing the detection results of the ability of specific T cells to secrete IFN-γ induced by tumor antigen epitope peptides. In FIGS. 5A and 5B, the experimental mice were euthanized, the monocytes derived from spleens were co-cultured with different peptides for 20 h to detect the secretion of IFN-γ by ELISPOT assay according to the manufacturer's instructions. Spleens were gently mashed and monocytes were isolated using a lymphocyte separation medium. The monocytes were incubated into wells of an ELISPOT plate and stimulated synchronously. The cells were incubated in an incubator at 37° C. and cytokines were captured. The number of T cells specifically secreting IFN-γ in splenocytes of the immunized mice can be detected by detecting the formation of spots. As can be seen from FIGS. 5A and 5B, the mice immunized with epitope peptides can induce T-cell immune responses associated with IFN-γ.

In conjunction with the above figures, the inhibitory effect of the vaccine on bladder cancer in mice was verified by in vivo experiments. In the mouse bladder cancer model, periodic vaccine treatment was started every 3 days on day 3 after tumor implantation, with a total of 7 times. As can be seen from the tumor growth curve and the solid tumor graph (FIG. 1), the vaccines containing the peptide fragments alleviated tumor growth to varying degrees compared with the control DPBS group and the CpG group. With the assistance of CpG, the combined KIF4A-1+KIF4A-2 vaccine and the NUF-2 vaccine significantly inhibited the growth of bladder cancer however, the CENPM vaccine and the combined IQGA3-1+IQGA3-2 vaccine did not show significant tumor inhibition. IFN-γ ELISPOT analysis was performed on mouse splenocytes. The results show that the peptide fragment CENPM and the peptide fragment NUF-2 can stimulate the splenocytes to produce significant T-cell responses compared with the control DPBS group and the CpG group.

In summary, the vaccine NUF-2 can cause the strongest T-cell response and the most significant tumor-inhibiting effects, demonstrating its potential as a potential tumor vaccine. It is worth noting that CENPM can induce T-cell responses but does not show strong anti-tumor effects; whereas the combined KIF4A-1+KIF4A-2 vaccine, which can effectively inhibit tumor growth, does not cause strong T-cell responses under separate stimulation of the peptide fragments, and it is speculated that multi-antigen vaccines are more effective in inhibiting cancer development than single antigen vaccines.

In the present disclosure, a plurality of genes co-expressed by tumor cells and stem cells were screened and the epitope peptides and extension peptides expressed by these genes were predicted and analyzed. By synthesizing these peptide fragments in vitro, these peptide fragments can effectively inhibit the growth of bladder cancer tumors while activating strong T cell-associated specific immune responses. Animal experiments have proved that these ESC-derived antigenic epitope peptide fragments have a good immunotherapeutic effect on bladder cancer.

The present disclosure, in conjunction with animal experiments, verifies the reliability of potential tumor-associated antigens TAAs and epitopes previously predicted by the bioinformatics algorithm based on the anti-tumor effect of ESCs as vaccines for the treatment of bladder cancer. The solution provides a complete set of procedures to verify its effectiveness from primary screening of tumor-specific antigens, preparation and vaccination of epitope peptide vaccines, and subsequent immunological experimental analysis. In addition, in the solution, individual peptide fragments CENPM, NUF-2, KIF4A-1, and KIF4A-2 having a cancer-inhibiting effect that are previously obtained and pre-experimentally validated are prepared into a polypeptide to be used as a vaccine to immunize mice, and the combination shows a good cancer-inhibiting effect. The disclosed technical route is easy to reproduce, shortens the cycle of discovery and verification, and greatly improves the application potential of stem cell-based gene epitope vaccines as therapeutic tumor vaccines in the context of precision medicine.

The above examples illustrate only several embodiments of the present disclosure and the descriptions thereof are more specific and detailed, but cannot therefore be construed as limiting the scope of the present disclosure. It should be noted that those of ordinary skill in the art may make several variations and improvements without departing from the concept of the present disclosure, and these variations and improvements fall within the protection scope of the present disclosure. Therefore, the protection scope of the present disclosure shall be subject to the appended claims.

What is claimed is:

1. Epitope peptides for tumor-associated antigens, derived from embryonic stem cells, wherein the tumor-associated antigens are selected from at least one of CENPM, IQGA3-1, IQGA3-2, KIF4A-1, KIF4A-2, and NUF-2.

2. The epitope peptides for tumor-associated antigens according to claim 1, comprising one of peptide fragments with amino acid sequences as shown in SEQ ID No. 1-SEQ ID No. 10.

3. The epitope peptides for tumor-associated antigens according to claim 2, comprising one of peptide fragments with amino acid sequences as shown in SEQ ID No. 11-SEQ ID No. 20.

4. Use of the epitope peptides for tumor-associated antigens according to claim 1 in the preparation of a medicament for inhibiting the growth of tumor cells and/or stimulating tumor cells to produce a T cell response.

5. The use according to claim 4, wherein the tumor comprises at least one of bladder cancer, esophageal cancer, kidney cancer, liver cancer, lung cancer, ovarian cancer, pancreatic cancer, prostate cancer, gastric cancer, and uterine cancer.

6. A medicament for treatment of tumors, comprising at least one of the epitope peptides for tumor-associated antigens according to claim 1.

7. The medicament for treatment of tumors according to claim 6, wherein the epitope peptide for tumor-associated antigens in the medicament for treatment of tumors comprises a peptide fragment having an amino acid sequence as shown in SEQ ID No. 10 or a peptide fragment having an amino acid sequence as shown in SEQ ID No. 20;
or, the epitope peptide for tumor-associated antigens in the medicament for treatment of tumors comprises a first peptide fragment comprising at least one of an amino acid sequence as shown in SEQ ID No. 9 and an amino acid sequence as shown in SEQ ID No. 19, and a second peptide fragment comprising at least one of an amino acid sequence as shown in SEQ ID No. 8 and an amino acid sequence as shown in SEQ ID No. 18.

8. The medicament for treatment of tumors according to claim 6, further comprising an adjuvant comprising at least one of CpG oligodeoxynucleotide and Poly IC.

9. The medicament for treatment of tumors according to claim 6, further comprising a pharmaceutically acceptable carrier and/or excipient.

10. Use of antigenic epitope peptides derived from embryonic stem cells in the preparation of a medicament for inhibiting the growth of tumor cells and/or stimulating tumor cells to produce a T cell response, wherein tumor-associated antigens are selected from at least one of CENPM, IQGA3-1, IQGA3-2, KIF4A-1, KIF4A-2, and NUF-2.


SEQUENCE LISTING (20 sequences)

SEQ ID NO: 1 - protein, 9 aa, synthetic construct
VHLAKSLPL

SEQ ID NO: 2 - protein, 9 aa, synthetic construct
RYFDALLKL

SEQ ID NO: 3 - protein, 9 aa, synthetic construct
VAPRYHLLL

SEQ ID NO: 4 - protein, 9 aa, synthetic construct
AYYFHLQTF

SEQ ID NO: 5 - protein, 9 aa, synthetic construct
AYQHLFYLL

SEQ ID NO: 6 - protein, 9 aa, synthetic construct
LHYTAAQLL

SEQ ID NO: 7 - protein, 10 aa, synthetic construct
WTYSFVASYV

SEQ ID NO: 8 - protein, 9 aa, synthetic construct
VFNTAVAPL

SEQ ID NO: 9 - protein, 10 aa, synthetic construct
TSFFSGCSPI

SEQ ID NO: 10 - protein, 11 aa, synthetic construct
FYMMPVNSEVM

SEQ ID NO: 11 - protein, 25 aa, synthetic construct
EDCASELKVHLAKSLPLPSSVNRPR

SEQ ID NO: 12 - protein, 25 aa, synthetic construct
EVEGENAQRYFDALLKLRQERGMGE

SEQ ID NO: 13 - protein, 25 aa, synthetic construct
GLDDVSLPVAPRYHLLLVAAKRQKA

SEQ ID NO: 14 - protein, 25 aa, synthetic construct
QHDMKDGTAYYFHLQTFQGIWEQPP

SEQ ID NO: 15 - protein, 25 aa, synthetic construct
KEKRQKLEAYQHLFYLLQTQPIYLA

SEQ ID NO: 16 - protein, 25 aa, synthetic construct
GKGKKQPSLHYTAAQLLEKGVLVEI

SEQ ID NO: 17 - protein, 25 aa, synthetic construct
AQELSGQGWTYSFVASYVEIYNETV

SEQ ID NO: 18 - protein, 25 aa, synthetic construct
DPSTEQEEVFNTAVAPLIKGVFKGY

SEQ ID NO: 19 - protein, 25 aa, synthetic construct
GKKKKRALASNTSFFSGCSPIEEEA

SEQ ID NO: 20 - protein, 25 aa, synthetic construct
YGIRLEHFYMMPVNSEVMYPHLMEG